SEQUENCE LISTING (5 sequences)

SEQ ID NO: 1 - protein, 460 aa, Homo sapiens
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 2 - protein, 219 aa, synthetic construct
IPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAQSKPERRRGLSWKSQNGRLYSIKSTKMLIHPTDSESFE

SEQ ID NO: 3 - protein, 493 aa, Homo sapiens
MRPLCVTCWWLGLLAAMGAVAGQEDGFEGTEEGSPREFIYLNRYKRAGESQDKCTYTFIVPQQRVTGAICVNSKEPEVLLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKYQHLATLAHNQSEIIAQLEEHCQRVPSARPVPQPPPAAPPRVYQPPTYNRIINQISTNEIQSDQNLKVLPPPLPTMPTLTSLPSSTDKPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 4 - protein, 233 aa, Homo sapiens
LTSLPSSTDKPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 5 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

(19) United States
(12) Patent Application Publication
(10) Pub. No.: US 2025/0012781 A1
(43) Pub. Date: Jan. 9, 2025

(54) METHODS FOR DETERMINING THE BIOLOGICAL ACTIVITY OF ANGPTL POLYPEPTIDES

(71) Applicant: Novartis AG, Basel (CH)

(72) Inventors: Christine Simone Aline HALLEUX, Dornach (CH); Carsten JACOBI, Muttenz (CH)

(21) Appl. No.: 18/709,345

(22) PCT Filed: Nov. 8, 2022
(86) PCT No.: PCT/IB2022/060727

§ 371 Date: May 10, 2024

Related U.S. Application Data
(60) Provisional application No. 63/277,852, filed on Nov. 10, 2021.

(51) Int. Cl. G01N 33/50

(52) U.S. Cl. CPC G01N 33/5023; G01N 33/5044

(57) ABSTRACT

Provided herein are methods for assaying the chondrogenesis-inducing activity of a therapeutic by measuring the expression and/or secretion levels of chondrogenesis biomarkers.

### TECHNICAL FIELD

The disclosures relates to methods for determining the biological activity of compounds capable of inducing chondrogenesis.

### BACKGROUND

Cartilage is formed through a process referred to as chondrogenesis. In vivo cartilage formation first involves the differentiation of mesenchymal stem cells into chondrocytes, which then secrete the molecules, e.g., collagen and proteoglycans, forming the extracellular matrix that comprises cartilage. Cartilage may be damaged much like any other tissues; however, unlike other tissues, cartilage has a very limited capacity for repair. The result is that damaged cartilage will progressively worsen over the years. Indeed, a hallmark of osteoarthritis (OA), the most common degenerative joint disease that affects more than 300 million people worldwide, is the progressive breakdown of articular cartilage. OA progression is mediated by both enzymatic degradation of the cartilage matrix and deficient cartilage matrix formation. Because the only therapeutic options to date are pain management and/or joint replacement, significant research efforts have been directed to improving cartilage self-repair by developing therapeutics capable of inducing chondrogenesis, e.g., by developing therapeutics that stimulate the differentiation of endogenous mesenchymal stem cells into chondrocytes, thereby leading to the regeneration of articular cartilage. However, the identification of chondrogenesis-inducing therapeutics can be challenging and time consuming. Thus, there is a need to develop methods capable of rapidly and accurately determining the chondrogenic-inducing activity of such therapeutics.

### SUMMARY

The present disclosure provides a method of determining the chondrogenesis-inducing activity of an ANGPTL polypeptide. In some embodiments, the method comprises exposing a cell culture to an ANGPTL polypeptide, measuring the expression and/or secretion level of a chondrogenesis biomarker, and comparing the expression and/or secretion level to a cell culture that has not been exposed to said ANGPTL polypeptide. In some embodiments, the expression and/or secretion level of the biomarker is increased as compared to a cell culture that has not been exposed to the ANGPTL polypeptide. In other embodiments, the expression and/or secretion level of the biomarker is decreased as compared to a cell culture that has not been exposed to the ANGPTL polypeptide.

In some embodiments, the ANGPTL polypeptide is ANGPTL2, ANTPTL3, ANGPTL4, or a derivative thereof. In a preferred embodiment, the ANGPTL polypeptide is SEQ ID NO: 2. In one embodiment, the cell culture used is comprised of chondrocyte cells. In certain embodiments, the chondrocyte cells are immortalized chondrocytes. In another embodiment, the chondrocyte cells are C-28/12 cells. In another embodiment, the cell culture is comprised of mesenchymal stem cells. In a preferred embodiment, the mesenchymal stem cells are human.

In one embodiment, the expression and/or secretion level of a chondrogenesis biomarker is measured by a method capable of quantifying the expression of chondrogenesis biomarker. In one embodiment, the method of quantifying the expression of a biomarker is an immunosorbent assay. In certain embodiments, the immunosorbent assay is ELISA or Western blotting. In another embodiment, the chondrogenesis biomarker is Annexin A6, CD44, CD151, ITM2A, FAM20B, FoxC1, FoxC2, SOX5, SOX6, SOX9, ACAN, Cathepsin B, CHAD, CHADL, Chondroadherin, Collagen II, Collagen IV, Collagen IX, CRTAC1, DSPG3, Decorin, IBSP/Sialoprotein II, Matrilin-1, Matrilin-3, Matrilin-4, MIA, Otoraplin/OTOR, URB, DKK1, FBN2, LEP, ALPL, CORIN, CLEC3b, or COMP. In a preferred embodiment, the chondrogenesis biomarker is DKK1.

In another embodiment, provided herein is a method of determining the activity of an ANGPTL polypeptide that induces chondrogenesis. In this embodiment, the method comprises adding an amount of the ANGPTL polypeptide to a cell culture and measuring the amount of DKK1 secretion, wherein the amount of DKK1 secretion following exposure to the polypeptide is increased as compared to the amount of DKK1 secretion of a cell culture wherein the polypeptide has not been added. In some embodiments, the ANGPTL polypeptide is ANGPTL2, ANGPTL3, ANGPTL4, or a derivative thereof. In a preferred embodiment, the ANGPTL polypeptide is SEQ ID NO: 2.

In another embodiment, provided herein is a method of determining the activity of an ANGPTL polypeptide that induces chondrogenesis. In this embodiment, the method comprises adding an amount of the ANGPTL polypeptide to a cell culture and measuring the amount of SOX9, ACAN, COMP, LEP, ALPL, CLEC3b, CORIN or FBN2 expression and/or secretion, (i) wherein the amount of SOX9, ACAN and COMP expression/secretion following exposure to the polypeptide is increased as compared to the amount of SOX9, ACAN and COMP expression/secretion of a cell culture wherein the polypeptide has not been added and (ii) wherein the amount of LEP, ALPL, CLEC3b, CORIN and FBN2 expression/secretion following exposure to the polypeptide is decrease as compared to the amount of LEP, ALPL, CLEC3b, CORIN and FBN2 expression/secretion of a cell culture wherein the polypeptide has not been added.

In some embodiments, when measuring the SOX9, ACAN, COMP, LEP, ALPL, CLEC3b, CORIN or FBN2 expression and/or secretion in the herein disclosed assays, the ANGPTL polypeptide is ANGPTL2, ANGPTL3, ANGPTL4, or a derivative thereof. In a preferred embodiment, the ANGPTL polypeptide is SEQ ID NO: 2.

In some embodiments, the provided herein is a method of identifying a substance that has a chondrogenesis-inducing effect comprising the steps of culturing cells capable of expressing chondrogenesis biomarkers, adding said substance to the cell culture, and measuring the secretion of chondrogenesis biomarkers following the addition of said substance. In one embodiment, the substance has a chondrogenesis-inducing effect if the level of chondrogenesis biomarkers markers following addition of the substance is altered as compared to the level of biomarkers in cell culture that has not been exposed to the substance. In certain embodiments, the level of chondrogenesis biomarkers is increased. In other embodiments, the level of chondrogenesis biomarkers is decreased. In certain embodiments, the chondrogenesis biomarker is Annexin A6, CD44, CD151, ITM2A, FAM20B, FoxC1, FoxC2, SOX5, SOX6, SOX9, ACAN, Cathepsin B, CHAD, CHADL, Chondroadherin, Collagen II, Collagen IV, Collagen IX, CRTAC1, DSPG3, Decorin, IBSP/Sialoprotein II, Matrilin-1, Matrilin-3, Matrilin-4, MIA, Otoraplin/OTOR, URB, DKK1, FBN2, LEP, ALPL, CORIN, CLEC3b, or COMP.

### DESCRIPTION OF THE DRAWINGS

FIG. 1. Compound 1 induces DKK1 (Dickkopf-related protein 1) secretion by chondrocytes. DKK1 ELISA of supernatants from C-28/I2 cells cultivated for 24 hours in C-28/I2 test medium supplemented with Compound 1. Levels are expressed in pg/ml. Values are mean+/−SD of measurements obtained from triplicate wells. One-way ANOVA with **** p<0.0001.

FIG. 2. DKK1 secretion is stimulated by Compound 1, ANGPTL3, ANGPTL2 and ANGPTL4 treatment in chondrocytes. DKK1 ELISA of supernatant from C-28/I2 cells cultivated for 24 hours in C-28/I2 test medium supplemented with Compound 1 or ANGPTLs. Toxicity scale based on cell morphology assessment in bright-field microscopy: + first slight sign of toxicity; ++: only few intact cells remaining; +++: many particles/cell debris present. Levels are expressed in pg/ml. Values are mean+/−SD of measurements obtained from triplicate wells. One-way ANOVA with **p<0.05, *** p<0.001 and **** p<0.0001.

FIG. 3. DKK1 secretion is stimulated by Compound 1 and truncated form of ANGPTL2 (C-terminal portion) in chondrocytes. Secretion of DKK1 by C-28/I2 cells upon Compound 1 and C-term ANGPTL2 (260-493) treatment. DKK1 ELISA of supernatant from C-28/I2 cells cultivated for 24 hours in C-28/I2 test medium supplemented with Compound 1 or C-term ANGPTL2. Toxicity scale based on cell morphology assessment in bright-field microscopy: + first slight sign of toxicity; ++: only few intact cells remaining; +++: many particles/cell debris present. Levels are expressed in pg/ml. Values are mean+/−SD of measurements obtained from triplicate wells. One-way ANOVA with **p<0.05, *** p<0.001 and **** p<0.0001.

FIG. 4. Compound 1 induces DKK1 secretion by human MSCs. DKK1 secretion, which accumulated between days 8 and 11, days 15 and 18, and days 22 and 25 of a 3D hMSC pellet culture, has been quantified by ELISA. Comparable effect of Compound 1 detected in hMSC from 2 different donors (out of 4 donors tested). Levels are expressed in pg/ml. Values are mean+/−SD of measurements obtained from triplicate wells. One-way ANOVA with *** p<0.001 and **** p<0.0001.

FIG. 5. Compound 1 induces production of lubricin protein by human MSCs. Lubricin immunohistochemical staining of human MSC pellets treated with increasing concentration of Compound 1 at 28 days. Isotype control shows the staining specificity; Bar=100 mm. Immunostaining quantification expressed in sum of lubricin intensity. Values are mean+/_SD of measurements of duplicate specimen obtained with cells isolated from one donor.

FIG. 6. Compound 1 reduces expression of alkaline phosphatase (ALPL) and leptin (LEP) transcript as well as leptin protein in human MSCs. Dose-response effect of Compound 1 on the relative expression (2-ΔCt) of alkaline phosphatase (ALPL) and leptin (LEP) and on the leptin (pg/ml) secreted in the supernatant (ELISA) in 3D pellets of hMSCs after 28 days of culture. Values are mean+/−SD of measurements obtained from triplicate wells. One-way ANOVA with **p<0.01, and **** p<0.0001.

FIG. 7. Compound 1 increases expression of COMP (Cartilage oligomeric matrix protein) in UE7T-13 cells. Dose-response effect of Compound 1 on the relative expression (2-ΔCt) of COMP transcript and secreted COMP protein (ng/ml, ELISA) in UE7T-13 cells cultivated in chondrogenic medium under inflammatory condition after 3, 7, 11 and 14 days of culture. Values are mean+/−SD of measurements obtained from triplicate wells. One-way ANOVA with *p<0.05, **p<0.01, *** p<0.001 and **** p<0.0001.

FIG. 8. Compound 1 increases expression of transcription factor SOX9 in UE7T-13. Dose-response effect of Compound 1 on the relative expression (2-ΔCt) of SOX9 transcript in UE7T-13 cells cultivated in chondrogenic medium under inflammatory condition after 3, 7, 11 and 14 days of culture. Values are mean+/−SD of measurements obtained from triplicate wells. One-way ANOVA with **p<0.01, *** p<0.001 and **** p<0.0001.

FIG. 9. Compound 1 increases expression of Acan (gene encoding Aggrecan, also known as cartilage-specific proteoglycan core protein or chondroitin sulfate proteoglycan 1) in UE7T-13. Dose-response effect of Compound 1 on the relative expression (2-ΔCt) of Acan transcript in UE7T-13 cells cultivated in chondrogenic medium under inflammatory condition after 3, 7, 11 and 14 days of culture. Values are mean+/−SD of measurements obtained from triplicate wells. One-way ANOVA with *** p<0.001 and **** p<0.0001.

### DETAILED DESCRIPTION

Modified human ANGPTL3 polypeptides previously have been shown to demonstrate chondrogenic and chondroprotective effects. Examples of such modified human ANGPTL3 polypeptides have been previously described in WO2014/138687, the contents of which are fully incorporated by reference. However, there remains a need for an improved method of determining the biological activity of such polypeptides to ensure they remain effective and/or retain potency following manufacturing, batch storage, stability testing, or any other instance where it is necessary to assess the biological activity of such polypeptides. The present inventors have discovered an effective method for fulfilling this need by developing an assay that measures the expression or secretion of biomarkers associated with chondrogenesis. In particular, the present inventors have discovered that DKK1 is an accurate and reliable indicator of the chondrogenesis-inducing activity of a given compound, such as a modified ANGPTL3 polypeptide.

As used herein, the terms “a” and “an” and “the” and similar references in the context of describing the invention are to be construed to cover both the singular and the plural, unless otherwise indicated herein or clearly contradicted by context. Where the plural form is used for compounds, salts, and the like, this is taken to mean also a single compound, salt, or the like.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“ANGPTL2” refers to a member of the angiopoietin protein family. An amino acid sequence of ANGPTL2 (GenBank Accession No. NP_036230.1) is set forth in SEQ ID NO: 3. “ANGPTL2 polypeptide” refers to a naturally occurring expressed polypeptide. For the purposes of the present disclosure, the numbering of an amino acid is typically determined with reference to the full-length wild-type human ANGPTL2 polypeptide sequence (SEQ ID NO: 3). Thus, in embodiments in which a polypeptide contains only a C-terminal portion of full-length ANGPTL2, but not the N-terminal portion, although the peptide is less than 493 amino acids in length, the numbering of the positions is based on SEQ ID NO: 3. For example, reference to position 350 of an ANGPTL2 polypeptide refers to position 350 of SEQ ID NO:3, even though the ANGPTL2 polypeptide itself may only be 200 amino acids in length. In determining an amino acid in a sequence of interest that “corresponds to” a position in a reference sequence, such as SEQ ID NO: 3, this is performed by optimally aligning the sequences, e.g., using the default CLUSTAL alignment parameters or default BLAST 2 alignment parameters and comparing the sequences. For example, position 350 in a sequence of interest that is “determined with reference to SEQ ID NO: 3”, or an amino acid that “corresponds to” position 350 of SEQ ID NO: 3, means the amino acid that aligns with position 350 of SEQ ID NO: 3, when the sequence of interest is optimally aligned with SEQ ID NO:3.

“ANGPTL3” refers to a member of the angiopoietin protein family. An amino acid sequence of ANGPTL3 (GenBank Accession No. NP_055310.1) is set forth in SEQ ID NO: 1. “ANGPTL3 polypeptide” refers to a naturally occurring expressed polypeptide. For the purposes of the present disclosure, the numbering of an amino acid is typically determined with reference to the full-length wild-type human ANGPTL3 polypeptide sequence (SEQ ID NO: 1). Thus, in embodiments in which a polypeptide contains only a C-terminal portion of full-length ANGPTL3, but not the N-terminal portion, although the peptide is less than 460 amino acids in length, the numbering of the positions is based on SEQ ID NO: 1. For example, reference to position 423 of an ANGPTL3 polypeptide refers to position 423 of SEQ ID NO:1, even though the ANGPTL3 polypeptide itself may only be 200 amino acids in length. In determining an amino acid in a sequence of interest that “corresponds to” a position in a reference sequence, such as SEQ ID NO: 1, this is performed by optimally aligning the sequences, e.g., using the default CLUSTAL alignment parameters or default BLAST 2 alignment parameters and comparing the sequences. For example, position 423 in a sequence of interest that is “determined with reference to SEQ ID NO: 1”, or an amino acid that “corresponds to” position 423 of SEQ ID NO: 1, means the amino acid that aligns with position 423 of SEQ ID NO: 1, when the sequence of interest is optimally aligned with SEQ ID NO: 1.

“ANGPTL4” refers to a member of the angiopoietin protein family. An amino acid sequence of ANGPTL4 (GenBank Accession No. NP_647475.1) is set forth in SEQ ID NO: 5. “ANGPTL4 polypeptide” refers to a naturally occurring expressed polypeptide. For the purposes of the present disclosure, the numbering of an amino acid is typically determined with reference to the full-length wild-type human ANGPTL4 polypeptide sequence (SEQ ID NO: 5). Thus, in embodiments in which a polypeptide contains only a C-terminal portion of full-length ANGPTL4, but not the N-terminal portion, although the peptide is less than 406 amino acids in length, the numbering of the positions is based on SEQ ID NO: 5. For example, reference to position 400 of an ANGPTL4 polypeptide refers to position 400 of SEQ ID NO:5, even though the ANGPTL4 polypeptide itself may only be 200 amino acids in length. In determining an amino acid in a sequence of interest that “corresponds to” a position in a reference sequence, such as SEQ ID NO: 5, this is performed by optimally aligning the sequences, e.g., using the default CLUSTAL alignment parameters or default BLAST 2 alignment parameters and comparing the sequences. For example, position 400 in a sequence of interest that is “determined with reference to SEQ ID NO: 5”, or an amino acid that “corresponds to” position 400 of SEQ ID NO: 5, means the amino acid that aligns with position 400 of SEQ ID NO: 5, when the sequence of interest is optimally aligned with SEQ ID NO:5.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “biologic activity” or “biologically active” refers to the ability of a compound to alter the physical, chemical, or biochemical properties of a biological system, pathway, tissue, cell, or molecule. Such an alteration can be, for example, an increase, decrease, maintenance, or modulation of the concentration or amount of a protein, polypeptide, peptide, DNA, RNA, saccharide, sugar, metabolite, precursor, cofactor, or other biological molecule whether in vivo, in vitro, or ex vivo. Such an alteration can be determined qualitatively or quantitatively.

The term “assay” or “assaying” is used to refer to the act of identifying, screening, probing or determining, which act may be performed by any conventional means. For example, a sample may be assayed for the presence of a particular biomarker by using an ELISA assay, a Western blot, a Northern blot, a Southern blot, mass spectroscopy, reverse transcribed quantitative polymerase chain reaction (RT-QPCR), imaging, etc., to detect whether that biomarker is present in the sample. Further, as used herein, the terms “assaying” and “determining” are used to mean testing and/or measuring.

Previous work with modified ANGPTL3 polypeptides, such as those described in WO2014/138687, the contents of which are fully incorporated by reference, have shown that such polypeptides have chondrogenic and chondroprotective effects. Compound 1 (SEQ ID NO: 2) is an example of such a modified ANGPTL3 polypeptide. The chondrogenesis-inducing effect of Compound 1 has been repeatedly established through in vitro studies, in vivo animal studies, and clinical trials. Despite the body of evidence establishing the chondrogenic effect of Compound 1, conventional assays for confirming such biological activity can be time consuming and difficult to evaluate. An improved method of determining the chondrogenic activity of such polypeptides was necessary. The inventors hypothesized that the relative expression levels of various biomarkers known to be involved in chondrogenesis could be utilized to develop the needed method.

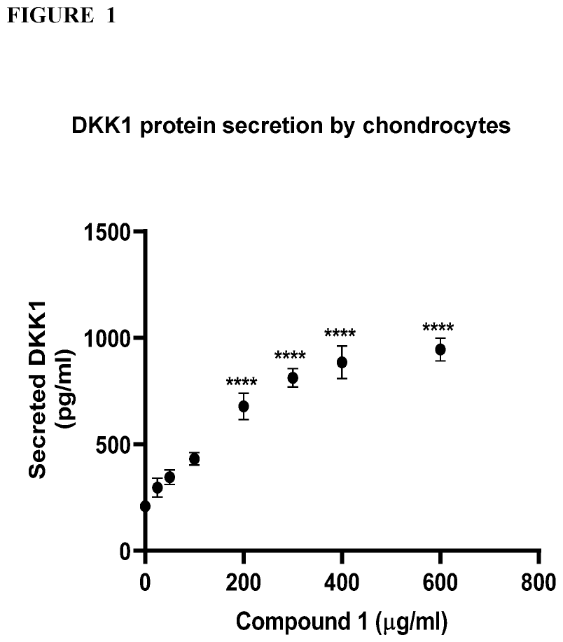

Chondrogenesis is a complex and dynamic cellular process involving the up- and down-regulation of a number of different genes along with an increase or decrease in the expression and/or secretion of various proteins. The alterations in these expression and/or secretion levels can be detected using a variety of standard techniques, and thus genes and/or proteins that have altered expression and/or secretion levels due to chondrogenesis have the potential to be used as biomarkers to detect the onset of chondrogenesis. The inventors determined that biomarkers that play a critical role in chondrogenesis, e.g., inducing chondrogenesis, inhibiting cartilage anabolic inhibitors, or inhibiting cartilage catabolic activity, have the potential to be reliable biomarkers for assaying chondrogenesis activity. For example, the WNT signaling pathway is thought to have an important role in OA progression because inhibition of WNT signaling has been reported to have cartilage anabolic and anti-hypertrophic effects, and therefore promote regeneration of hyaline cartilage. DKK1 is a known inhibitor of WNT signaling. DKK1 also has been shown to be negatively correlated to OA severity, e.g., DKK1 overexpression in chondrocytes inhibits experimental OA cartilage destruction in mice. DKK-1 also inhibits hypertrophic differentiation and ILI1β-induced MEMP expression in articular chondrocytes, which may contribute to the development of OA. The inventors thus hypothesized that DKK1 secretion levels could act as a surrogate for assaying the chondrogenic activity of a compound, e.g., Compound 1. As shown in FIG. 1, DKK1 secretion increased following exposure to Compound 1 in a dose-dependent manner, which indicates that DKK1 can be used as a surrogate marker to determine chondrogenesis activity.

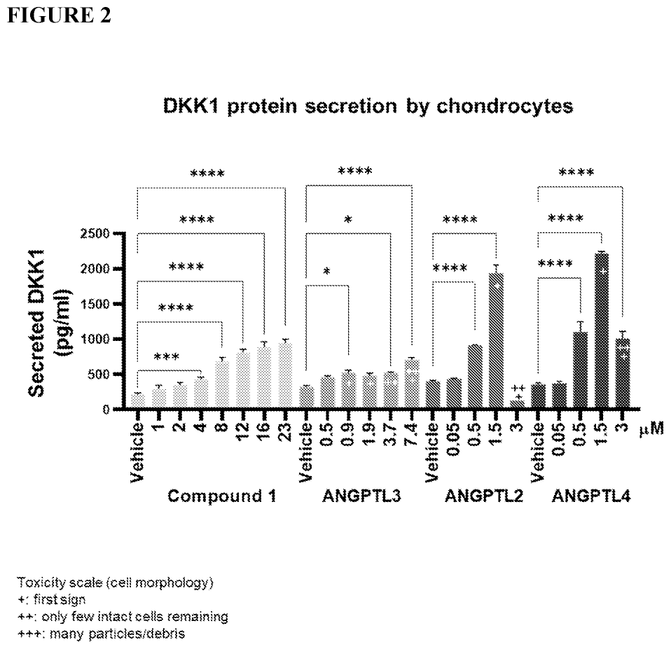

Other members of the ANGPTL protein family that have been reported to be involved in chondrogenesis and cartilage remodeling, namely ANGPTL2 and ANGPTL4, were also evaluated. Comparing Compound 1 with ANGPTL3, ANGPTL2 and ANGPTL4 in the assay, it was observed that all of these molecules have the ability to induce the secretion of DKK1 into the supernatant. (FIG. 2). However, unlike Compound 1, the ANGPTL2 and ANGPTL4 proteins also induced cytotoxicity at low concentrations. The results of this experiment validate the robustness of the assay described herein and indicate that it may be used to quickly screen ANGPTL polypeptides for potential chondrogenic activity.

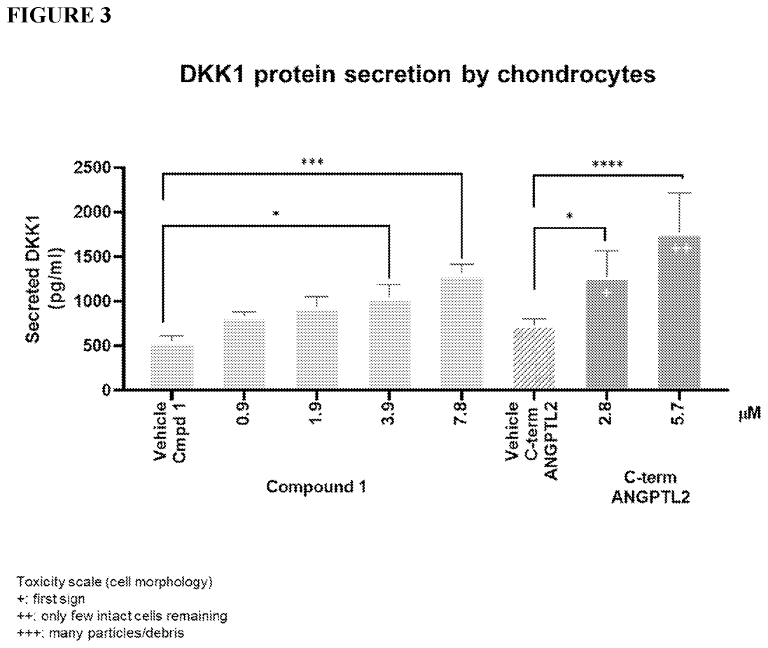

The assay described herein has also been used to screen a truncated form of ANGPTL2 (FIG. 3). The results of this experiment demonstrate that C-term ANGPTh2 (260-493; SEQ ID NO:4) is able to induce DKK1 secretion level comparable to Compound 1. This suggests that truncated ANGPTh2 retains chondrogenic potential and could potentially be developed as a therapeutic to induce cartilage repair.

TABLE 1
Amino Acid Sequences

| SEQ | | |
|---|---|---|
| ID | Description | Amino Acid Sequence |
| 1 | ANGPTL3 | mftiklllfi vplvissrid qdnssfdsls pepksrfaml ddvkilangl |
| | | lqlghglkdf vhktkgqind ifqklnifdq sfydlslqts eikeeekelr |
| | | rttyklqvkn eevknmslel nsklesllee killqqkvky leeqltnliq |
| | | nqpetpehpe vtslktfvek qdnsikdllq tvedqykqln qqhsqikeie |
| | | nqlrrtsiqe pteislsskp raprttpflq lneirnvkhd gipaecttiy |
| | | nrgehtsgmy airpsnsqvf hvycdvisgs pwtlighrid gsqnfnetwe |
| | | nykygfgrld gefwlgleki ysivkqsnyv lrieledwkd nkhyieysfy |
| | | lgnhetnytl hlvaitgnvp naipenkdlv fstwdhkakg hfncpegysg |
| | | gwwwhdecge nnlngkynkp rakskperrr glswksqngr lysikstkml |
| | | ihptdsesfe |
| 2 | Compound | ipaecttiyn rgehtsgmya irpsnsqvfh vycdvisgsp wtliqhridg |
| | 1 | sqnfnetwen ykygfgrldg efwlglekiy sivkqsnyvl rieledwkdn |
| | | khyieysfyl gnhetnytlh lvaitgnvpn aipenkdlvf stwdhkakgh |
| | | fncpegysgg wwwhdecgen nlngkynkpr aqskperrrg lswksqngrl |
| | | ysikstkmli hptdsesfe |
| 3 | ANGPTL2 | mrplcvtcww lgllaamgav agqedgfegt eegsprefiy lnrykrages |
| | | qdkctytfiv pqqrvtgaic vnskepevll enrvhkqele linnellkqk |
| | | rqietlqqlv evdggivsev kllrkesrnm nsrvtqlymq llheiirkrd |
| | | nalelsqlen rilnqtadml qlaskykdle hkyqhlatla hnqseiiaql |
| | | eehcqrvpsa rpvpqpppaa pprvyqppty nriinqistn eiqsdqnlkv |
| | | lppplptmpt ltslpsstdk psgpwrdclq aledghdtss iylvkpentn |
| | | rlmqvwcdqr hdpggwtviq rrldgsvnff rnwetykqgf gnidgeywlg |
| | | leniywltnq gnykllvtme dwsgrkvfae yasfrlepes eyyklrlgry |
| | | hgnagdsftw hngkqfttld rdhdvytgnc ahyqkggwwy nacahsnlng |
| | | vwyrgghyrs ryqdgvywae frggsyslkk vvmmirpnpn tfh |
| 4 | C-Term | ltslpsstdk psgpwrdclq aledghdtss iylvkpentn rlmqvwcdqr |
| | ANGPTL2 | hdpggwtviq rrldgsvnff rnwetykqgf gnidgeywlg leniywltnq |
| | | gnykllvtme dwsgrkvfae yasfrlepes eyyklrlgry hgnagdsftw |
| | | hngkqfttld rdhdvytgnc ahyqkggwwy nacahsnlng vwyrgghyrs |
| | | ryqdgvywae frggsyslkk vvmmirpnpn tfh |
| 5 | ANGPTL4 | msgaptagaa lmlcaatavl lsaqggpvqs ksprfaswde mnvlahgllq |
| | | lgqglrehae rtrsqlsale rrlsacgsac qgtegstdlp lapesrvdpe |
| | | vlhslqtqlk aqnsriqqlf hkvaqqqrhl ekqhlriqhl qsqfglldhk |
| | | hldhevakpa rrkrlpemaq pvdpahnvsr lhrlprdcqe lfqvgerqsg |
| | | lfeiqpqgsp pflvnckmts dggwtviqrr hdgsvdfnrp weaykagfgd |
| | | phgefwlgle kvhsitgdrn srlavqlrdw dgnaellqfs vhlggedtay |
| | | slqltapvag qlgattvpps glsvpfstwd qdhdlrrdkn cakslsggww |
| | | fgtcshsnln gqyfrsipqq rqklkkgifw ktwrgryypl qattmliqpm |
| | | aaeaas |

The above experiments demonstrate that DKK1 may be used as a surrogate biomarker to assess the biological activity of ANGPTL polypeptides, e.g., Compound 1. Other biomarkers that are implicated in chondrogenesis are also suitable for use in the presently disclosed methods. Non-limiting examples of such biomarkers include Annexin A6, CD44, CD151, ITM2A, FAM20B, FoxC1, FoxC2, SOX5, SOX6, SOX9, ACAN, Cathepsin B, CHAD, CHADL, Chondroadherin, Collagen II, Collagen IV, Collagen IX, CRTAC1, DSPG3, Decorin, IBSP/Sialoprotein II, Matrilin-1, Matrilin-3, Matrilin-4, MIA, Otoraplin/OTOR, URB, DKK1, FBN2, LEP, ALPL, CORIN, CLEC3b, or COMP. A person of skill in the art will further recognize that other biomarkers shown to be correlated with chondrogenic activity are also suitable for use in the disclosed assay and methods.

The assay and methods provided herein may also be used to screen and/or identify potential therapeutics with chondrogenic-inducing activity. A person of ordinary skill in the art will recognize that different compounds may induce chondrogenesis via different pathways or mechanisms. Thus, in some embodiments, multiple biomarkers may be used to screen potential therapeutics for chondrogenesis-inducing activity. It will be appreciated that the biomarkers used to screen one potential therapeutic may be the same or different from biomarkers used to screen another potential therapeutic.

Conventional methods of detecting and quantitating the selected biomarker, e.g., DKK1, may be used with the presently disclosed methods. Examples of such conventional methods include immunoassays, such as enzyme-linked immunoabsobent assay (ELISA) or Western blot analysis, and mass spectrometry. However, a person of ordinary skill in the art would recognize that any method capable of detecting a specific protein in a cell culture is acceptable for use with the described method. In some embodiments the detection method is ELISA, and in other embodiments the method is a sandwich ELISA.

Methods of detecting gene expression are also suitable for use in the disclosed assay and methods. Non-limiting examples of methods for detecting gene expression including PCR, quantitative PCR, RNA-seq analysis, or microarrays. A person of ordinary skill will further understand that other methods for detecting nucleic acid levels are also acceptable.

Chondrocytes are suitable for use with the assay and methods disclosed herein because chondrocytes are already further differentiated into cells capable of expressing typical chondrogenesis markers and secreting extracellular matrix proteins that comprise cartilage. Because chondrocytes are further differentiated, the biological activity of chondrogenesis-inducing or hypertrophy inhibitory compounds being assayed may be more rapidly determined when compared to non-differentiated cells. In addition, a chondrocyte cell line may be expanded many times, which makes it possible to work with the same batch of cells. Examples of chondrocyte cell lines that may be used with the present methods include, but are not limited to, C-28/I2 and T/C28a2.

Mesenchymal stem cells are also suitable for use with the present methods. Mesenchymal stem cells are primary cells isolated from the bone marrow. In some embodiments the mesenchymal stem cells are human. These cells are not yet differentiated, and must be cultivated under chondrogenic conditions for some time to reach a stage of differentiation that is comparable to chondrocytes. During that differentiation process down the chondrogenic lineage, the mesenchymal stem cells progressively become chondrocytes that express typical chondrogenic markers and are able to secrete extra cellular matrix proteins encountered in cartilage. Therefore, assays on chondrocytes derived from mesenchymal stem cells take longer to complete as compared to assays on chondrocytes. In addition, human mesenchymal stem cells may only be expanded for a few passages before losing the ability to differentiate into different cell lineages. However, an advantage of the chondro-differentiated mesenchymal stem cells is their closer resemblance to primary chondrocytes.

Although chondrocytes and mesenchymal stem cells are well suited for use in the disclosed assay and methods, a person of ordinary skill will recognize that any cell capable of expressing a chondrogenesis-related marker, in particular DKK1, may be used.

The present method provides a rapid and effective method of determining the biological activity of a compound with chondrogenesis-inducing potential. In some embodiments, the compound is a potential therapeutic. In other embodiments, the compound is an ANGPTL polypeptide or variant. In another embodiment, the compound is Compound 1.

In one aspect, the present method involves measuring the expression and/or secretion of a biomarker after exposing a cell culture to a compound with chondrogenesis-inducing activity. In some embodiments, a baseline expression level of the biomarker is established prior to exposure to the compound. In other embodiments, the biomarker expression level is compared to a control that has not been exposed to a compound.

In another aspect, the present method involves determining whether a compound has chondrogenesis-inducing activity by measuring the expression and/or secretion of biomarkers after exposing a cell culture to a compound. In some embodiments, the expression and/or secretion of the biomarker is increased. In other embodiments, the expression and/or secretion of the biomarker is decreased.

In another aspect, the biomarker used in this assay is a biomarker that is associated with chondrogenesis. In another aspect, the biomarker is associated with the inhibition of cartilage anabolic activity. In some embodiments, the biomarker is Annexin A6, CD44, CD151, ITM2A, FAM20B, FoxC1, FoxC2, SOX5, SOX6, SOX9, ACAN, Cathepsin B, CHAD, CHADL, Chondroadherin, Collagen II, Collagen IV, Collagen IX, CRTAC1, DSPG3, Decorin, IBSP/Sialoprotein II, Matrilin-1, Matrilin-3, Matrilin-4, MIA, Otoraplin/OTOR, URB, DKK1, FBN2, LEP, ALPL, CORIN, CLEC3b, or COMP. In another embodiment, the biomarker is DKK1.

In another aspect, the cell culture is comprised of chondrocytes. In other embodiments, the cell culture is composed of mesenchymal stem cells. In another embodiment, the mesenchymal stem cells are human.

In another aspect, the biomarker expression and/or secretion level in a cell culture is determined through a detection method capable of quantitatively or qualitatively measuring protein and/or gene expression/secretion. In some embodiments, the detection method is Western blotting, or mass spectroscopy. In another embodiment, the detection method is ELISA, and in yet another embodiment the detection method is a sandwich ELISA.

In another aspect, the present method may be used during the manufacturing process to confirm a compound possesses chondrogenesis-inducing activity. Thus, in one aspect, the present disclosure is directed to a method of validating the activity of a compound with chondrogenic activity prior to release. In another aspect, the present method may be used to determine the chondrogenesis activity of a compound following storage. In yet another aspect, the present method may be used to determine the chondrogenesis-inducing activity of a compound prior to administration to a patient.

In another aspect, the described assay and methods may be used to screen compounds for potential chondrogenesis-inducing activity. In one embodiment, a compound with potential chondrogenesis-inducing activity is exposed to a cell culture and the expression and/or secretion levels of chondrogenesis biomarkers is measured. In some embodiments, the biomarker levels are increased as compared to cell culture that has not been exposed to the compound with potential chondrogenesis-inducing activity. In another embodiment, the biomarker levels are decreased as compared to a cell culture that has not been exposed to the compound with potential chondrogenesis-inducing activity

### Example 1: DKK1 ELISA Assay

Multi-array High-Bind 96-well plates (MSD) were pre-coated overnight at 4° C. with a monoclonal anti-human Dkk1 (1 mg/ml PBS) antibody (R&D), followed by blocking with 1% Casein TBS (BioRad) for 1 h in a Thermomixer at 450 rpm and 4 washing steps with 0.5×TBST (Sigma) at RT. Cell supernatants were added in the appropriate wells and incubated at RT in a Thermomixer for 1 h at 450 rpm, followed by 4 washing steps and the addition of biotinylated anti-human DKK1 (R&D) incubated at RT in a Thermomixer for 1 h at 450 rpm. After 4 washing steps, a streptavidin sulfo-TAG solution (MSD) was added and incubated at RT in a Thermomixer for 30 min at 450 rpm. Four additional washing steps were performed prior to the addition of 2× Read Buffer (MSD) for detection of the electrochemiluminescence counts with a MSD Sector S600 reader. ELISAs were performed on cell culture supernatants stored at −80° C.

TABLE 2
Reagents used for immunocytochemistry and ELISA

| Description | Company | Catalog number | Concentration | |
|---|---|---|---|---|
| Anti-human DKK1 | R&D | MAB10962 | 2 | ug/ml |
| Biotinylated | R&D | BAF1096 | 0.25 | ug/ml |
| Anti-Human DKK1 | | | | |

### Example 2: C-28/12 Cell Culture for Detection of DKK1 Secretion Induced by Compound 1

The human chondrocyte cell line C-28/I2 (licensed from Dr. Mary Goldring, Massachusetts General Hospital, Boston, USA) was expanded in DMEM/F12 medium containing 10% FCS (Millipore), 50 μg/ml L-ascorbic acid 2 phosphate (Wako Pure Chemical), 100 IU/ml Penicillin and 100 μg/ml Streptomycin. To test Compound 1 activity, cells were seeded at 50′000 cells/well (96-well plate format, Costar) in 100 μl DMEM/F12 medium with 1% FCS (Millipore), 50 μg/ml L-ascorbic acid phosphate (Wako Pure Chemical), 100 IU/ml Penicillin, 100 μg/ml Streptomycin supplemented with Compound 1 (Novartis) at the concentrations specified in the figures, or vehicle control, and incubated for 24 h. Cell cultures were performed at 37° C. in a humidified incubator with 5% C02.

A dose-dependent increase of DKK1 protein in supernatants of C-28/I2 cells up to 4.5-fold over control was observed. This happened within 24 h of Compound 1 treatment. (FIG. 1).

### Example 3: C-28/12 Cell Culture for Detection of DKK1 Secretion Induced by ANGPTL Variants

The human chondrocyte cell line C-28/I2 (licensed from Dr. Mary Goldring, Massachusetts General Hospital, Boston, USA) was expanded in DMEM/F12 medium containing 10% FCS (Millipore), 50 μg/ml L-ascorbic acid 2 phosphate (Wako Pure Chemical), 100 IU/ml Penicillin and 100 μg/ml Streptomycin. To test Compound 1 and ANGPTL variants activity, cells were seeded at 50′000 cells/well (96-well plate format, Costar) in 100 μl DMEM/F12 medium with 1% FCS (Millipore), 50 μg/ml L-ascorbic acid phosphate (Wako Pure Chemical), 100 IU/ml Penicillin, 100 μg/ml Streptomycin supplemented with Compound 1 (Novartis), ANGPTL2 (R&D), ANGPTL3 (Novartis) or ANGPTL4 (R&D) at the concentrations specified in the figures, or vehicle control, and incubated for 24 h. Cell cultures were performed at 37° C. in a humidified incubator with 5% C02.

ANGPTL2 and ANGPTL4, the two ANGPTL family members expressed in cartilage and previously reported to affect chondrogenesis and cartilage matrix remodelling, respectively, were about 20-fold more potent than Compound 1 in stimulating DKK1 secretion. (FIG. 2). However, ANGPTL2, ANGPTL3 and ANGPTL4 induced strong cytotoxicity above 3 μM concentrations and therefore were tested at lower concentrations than Compound 1. No cytotoxicity with Compound 1 was observed (FIG. 2).

### Example 4: C-28/12 Cell Culture for Detection of DKK1 Secretion Induced by a Truncated ANGPTL Variant

The human chondrocyte cell line C-28/I2 (licensed from Dr. Mary Goldring, Massachusetts General Hospital, Boston, USA) was expanded in DMEM/F12 medium containing 10% FCS (Millipore), 50 μg/ml L-ascorbic acid 2 phosphate (Wako Pure Chemical), 100 IU/ml Penicillin and 100 μg/ml Streptomycin. To test Compound 1 and truncated ANGPTL variant activity, cells were seeded at 50′000 cells/well (96-well plate format, Costar) in 100 μl DMEM/F12 medium with 1% FCS (Millipore), 50 μg/ml L-ascorbic acid phosphate (Wako Pure Chemical), 100 IU/ml Penicillin, 100 μg/ml Streptomycin supplemented with Compound 1 (Novartis) or C-term ANGPTL2 (R&D) at the concentrations specified in the figures, or vehicle control, and incubated for 24 h. Cell cultures were performed at 37° C. in a humidified incubator with 5% C02.

C-term ANGPTL2 (260-493; SEQ ID NO: 4) the truncated form of an ANGPTL family member expressed in cartilage and previously reported to affect chondrogenesis was as potent as Compound 1 in stimulating DKK1 secretion. (FIG. 3). However, the truncated form of ANGPTL2 induced strong cytotoxicity above 3 μM concentrations. No cytotoxicity with Compound 1 was observed (FIG. 3).

### Example 5: hMSCs Cell Culture for Detection of Chondrogenesis Biomarker Expression Induced by Compound 1

For 3D pellet culture assays, bone marrow-derived human Mesenchymal Stem Cells (hMSCs) from 4 different donors (Lonza Verviers, Belgium) were first expanded for 2 passages in Lonza medium MSCGM-BulletKit™ and stored in liquid nitrogen. Cells were further expanded in DMEM 1 g/L glucose, 10% FBS, 6 mM L-glutamine, 10 mM HEPES, 50 IU/ml Penicillin, 50 g/ml Streptomycin and 1 ng/ml human basic FGF (R&D Systems). For 3D cultures, passage 6 cells were seeded at 3.5×105 cells/well in 96-well V-bottom plates (Costar), sedimented by centrifugation (5 min, 250 g) and cultured for 4 weeks in DMEM high glucose, 0.125% BSA (Sigma), ITS (6.25 μg/ml human insulin, 6.25 μg/ml human transferrin, 6.25 ng/ml sodium selenite, Roche), 5.3 μg/ml linoleic acid (Sigma), 50 μg/ml L-ascorbic acid phosphate (AA, Wako Pure Chemical), 100 ng/ml dexamethasone (Sigma), 40 μg/ml proline (Sigma), 100 IU/ml Penicillin and 100 μg/ml Streptomycin supplemented, as indicated, with LNA043 (recombinantly expressed in CHO cells, Novartis) or vehicle control. Medium was changed 3 times per week. Cell cultures were performed at 37° C. in a humidified incubator with 5% C02.

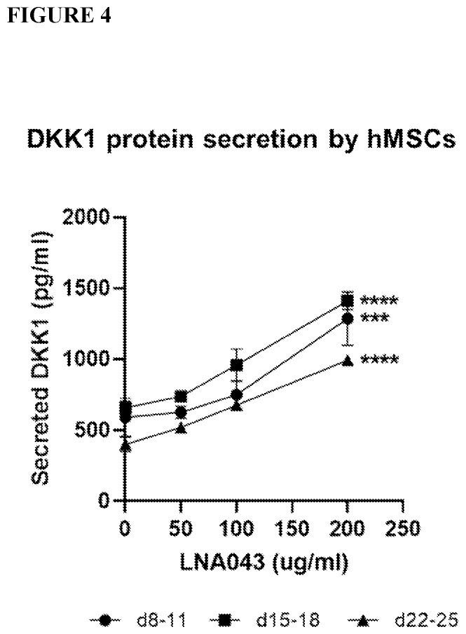

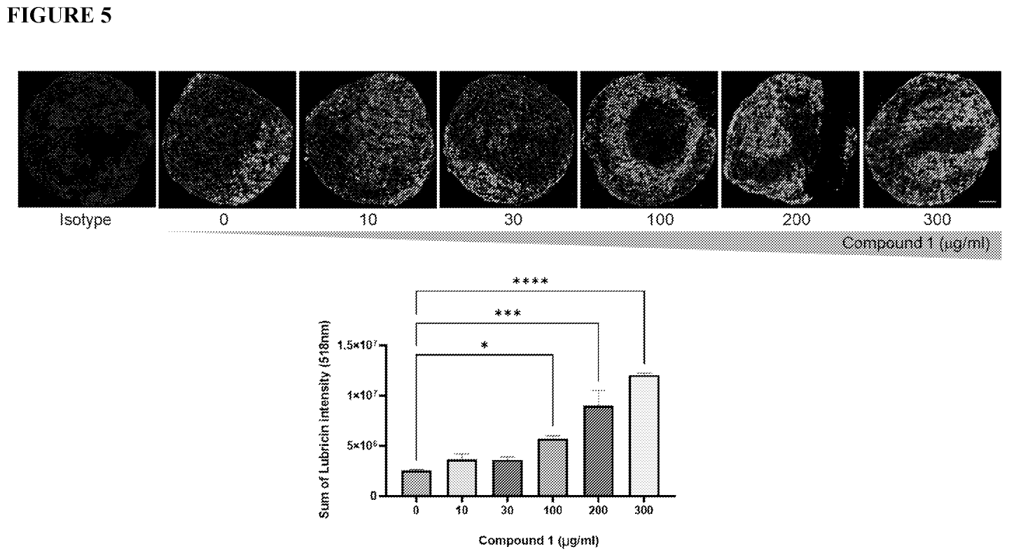

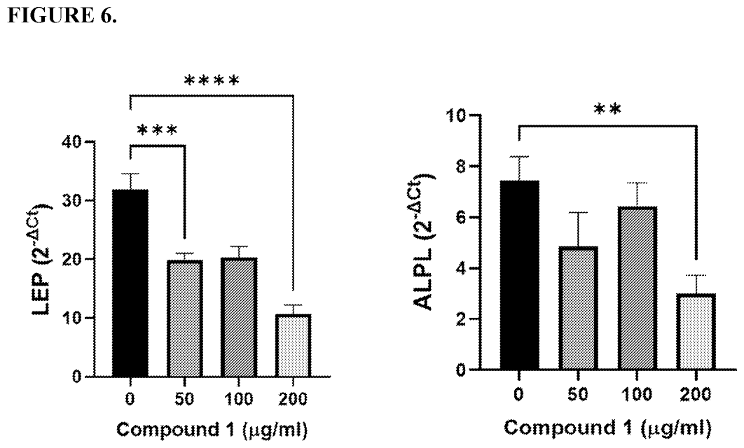

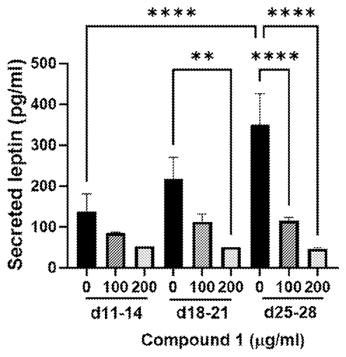

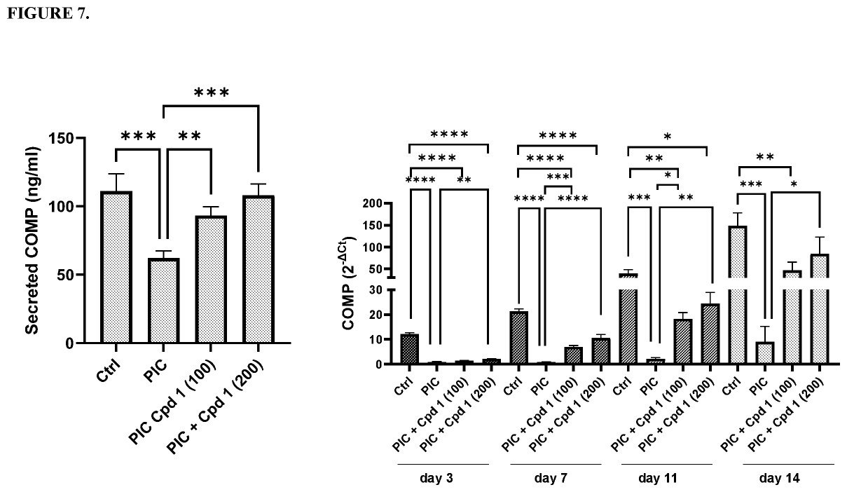

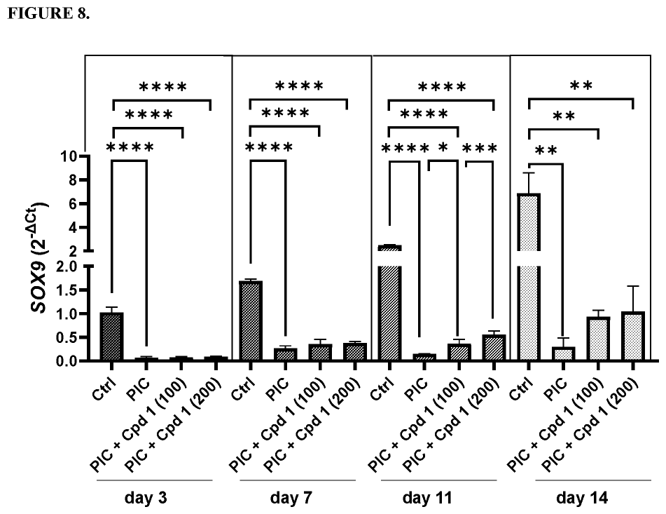

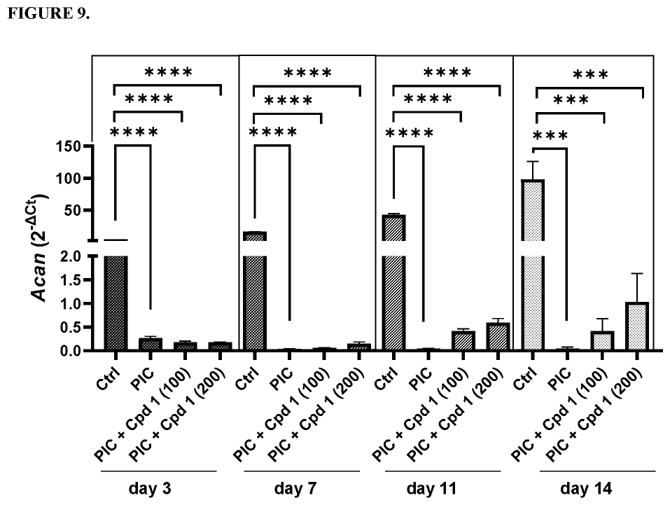

Compound 1 (SEQ ID NO: 2) treatment during 28 days of chondrogenic differentiation induced a dose-dependent up-regulated secretion of WNT inhibitor protein DKK1 in supernatants. (FIG. 4). In addition, Compound 1 treatment during 28 days of chondrogenic differentiation induced a dose-dependent, up to 5.7-fold increase in expression of the cartilage superficial zone glycoprotein lubricin/proteoglycan 4 (PRG4) detected by immunohistochemical staining of the 3D pellets. (FIG. 5). At the gene expression level, Compound 1 decreased cartilage hypertrophy marker alkaline phosphatase and the pro-inflammatory adipokine leptin. (FIG. 6). Additional genes that have been shown to be downregulated by Compound 1 include FBN2, CORIN, and CLEC3b, while COMP gene has been shown to be upregulated (data not shown).

### Example 6: Chondrogenesis Biomarkers Regulated by Compound 1

TABLE 3
Gene and protein expression regulated by Compound 1 in the
3D hMSC and the ihMSC (UE7T-13 cell) RR assay, respectively

| | Gene | Protein | Assay |
|---|---|---|---|
| DKK1 | | increase | 3D hMSC |
| SOX9 | increase | not secreted | UE7T-13(inflammatory |
| | | | condition) |
| Acan | increase | not determined | UE7T-13 (inflammatory |
| | | | condition) |
| COMP | increase | increase | UE7T-13 (inflammatory |
| | | | condition) |
| LEP | decrease | decrease | 3D hMSC |
| ALPL | decrease | not determined | 3D hMSC |
| CLEC3b | decrease | not determined | 3D hMSC |
| CORIN | decrease | not determined | 3D hMSC |
| FBN2 | decrease | not determined | 3D hMSC |

Having thus described several aspects of several embodiments, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

Those skilled in the art will recognize, or be able to ascertain, using no more than routine experimentation, numerous equivalents to the specific embodiments described specifically herein.

Such equivalents are intended to be encompassed in the scope of the following claims.

1. A method of determining the chondrogenesis-inducing activity of an ANGPTL polypeptide comprising exposing a cell culture to said ANGPTL polypeptide, measuring the expression and/or secretion level of a chondrogenesis biomarker, and comparing the expression and/or secretion level to a cell culture that has not been exposed to said ANGPTL polypeptide.

2. The method of claim 1, wherein said ANGPTL polypeptide is SEQ ID NO: 2.

3. The method of claim 1-2, wherein said cell culture is comprised of chondrocyte cells.

4. The method of claim 1-2, wherein said cell culture is comprised of mesenchymal stem cells.

5. The method of claim 4, wherein said mesenchymal stem cells are human.

6. The method of claim 1-5, wherein said expression and/or secretion level of a chondrogenesis biomarker is measured by an immunosorbent assay.

7. The method of claim 1-6, wherein said immunosorbent assay is ELISA or Western blotting.

8. The method of claim 1-7, wherein said biomarker is Annexin A6, CD44, CD151, ITM2A, FAM20B, FoxC1, FoxC2, SOX5, SOX6, SOX9, ACAN, Cathepsin B, CHAD, CHADL, Chondroadherin, Collagen II, Collagen IV, Collagen IX, CRTAC1, DSPG3, Decorin, IBSP/Sialoprotein II, Matrilin-1, Matrilin-3, Matrilin-4, MIA, Otoraplin/OTOR, URB, DKK1, FBN2, LEP, ALPL, CORIN, CLEC3b, or COMP

9. The method of claim 8, wherein said biomarker is DKK1.

10. The method of claim 1-9, wherein the expression and/or secretion level of the biomarker DKK1, SOX9, ACAN or COMP is increased compared to a cell culture that has not been exposed to the ANGPTL polypeptide.

11. The method of claim 1-9, wherein the expression and/or secretion level of the biomarker LEP, ALPL, CLEC3b, CORIN or FBN2 is decreased compared to a cell culture that has not been exposed to the ANGPTL polypeptide.

12. A method of determining the biological activity of an ANGPTL polypeptide that induces chondrogenesis comprising adding an amount of the polypeptide to a cell culture and measuring the amount of DKK1 secretion, wherein an increase in DKK1 secretion following exposure to the polypeptide is increased as compared to the DKK1 secretion of a cell culture wherein the polypeptide has not been added.

13. The method of claim 12, wherein the ANGPTL polypeptide is ANGPTL2, ANGPTL3, or ANGPTL4.

14. The method of claim 12, wherein the compound is an ANGPTL3 derivative.

15. The method of claim 14, wherein the compound is SEQ ID NO: 2.

16. The method of claim 12, wherein the compound is an ANGPTL2 derivative.

17. The method of claim 16, wherein the compound is SEQ ID NO: 4.

18. The method of claim 12, wherein the compound is an ANGPTL4 derivative.

19. A method of identifying a substance that has a chondrogenesis-inducing effect comprising the steps of:
a. Culturing cells capable of expressing chondrogenesis biomarkers;
b. Adding said substance to the cell culture;
c. Measuring the secretion of chondrogenesis biomarkers following the addition of said substance;
wherein the substance has a chondrogenesis-inducing effect if the level of chondrogenesis biomarkers following addition of the substance is altered as compared to the level of biomarkers in cell culture that has not been exposed to the substance.

20. The method of claim 19, wherein said level of chondrogenesis biomarkers is increased.

21. The method of claim 19, wherein said level of chondrogenesis biomarkers is decreased.

22. The method of claim 20-21, wherein said chondrogenesis biomarker is Annexin A6, CD44, CD151, ITM2A, FAM20B, FoxC1, FoxC2, SOX5, SOX6, SOX9, ACAN, Cathepsin B, CHAD, CHADL, Chondroadherin, Collagen II, Collagen IV, Collagen IX, CRTAC1, DSPG3, Decorin, IBSP/Sialoprotein II, Matrilin-1, Matrilin-3, Matrilin-4, MIA, Otoraplin/OTOR, URB, DKK1, FBN2, LEP, ALPL, CORIN, CLEC3b, or COMP.
